(19) United States
(12) Patent Application Publication
(10) Pub. No.: US 2025/0170190 A1
(43) Pub. Date: May 29, 2025

(54) STRAIN OF ENTEROCOCCUS FAECALIS, COMPOSITE BACTERIAL AGENT AND APPLICATIONS THEREOF

(71) Applicant: HEALTH AND HAPPINESS (H&H) HONG KONG LIMITED, Hong Kong (CN)

(72) Inventors: Lingling ZHAO, Hong Kong (CN); Hua ZHANG, Hong Kong (CN); Ying LI, Hong Kong (CN)

(73) Assignee: HEALTH AND HAPPINESS (H&H) HONG KONG LIMITED, Hong Kong (CN)

(21) Appl. No.: 19/028,716

(22) Filed: Jan. 17, 2025

Related U.S. Application Data
(63) Continuation of application No. PCT/CN2023/128876, filed on Oct. 31, 2023.

(30) Foreign Application Priority Data
Oct. 13, 2023 (CN) 202311330221.0

(51) Int. Cl. A61K 35/744; A61K 35/745; A61P 1/00

(52) U.S. Cl. CPC A61K 35/744; A61K 35/745; A61P 1/00

(57) ABSTRACT

A strain of Enterococcus faecalis, a composite bacterial agent, and application thereof, which relates to the field of microbiology. The Enterococcus faecalis strain was deposited on Oct. 13, 2022, at the Guangdong Microbial Culture Collection Center (GDMCC) with the deposit number GDMCC No. 62869. Both the Enterococcus faecalis strain and the composite bacterial agent can effectively treat intestinal disorders and inflammatory bowel disease in felines and can be used in the preparation of related pharmaceuticals.

### CROSS-REFERENCE TO RELATED APPLICATIONS

The present application is a Bypass Continuation Application of PCT Patent Application No. PCT/CN2023/128876, filed on Oct. 31, 2023, which claims priority to Chinese Patent Application No. 202311330221.0, filed on Oct. 13, 2023. The content of all aforesaid applications is incorporated herein by reference

### FIELD

The present disclosure relates to the field of microorganisms, in particular to a strain of Enterococcus faecalis, a composite bacterial agent and applications thereof.

### BACKGROUND

Intestinal diseases are a common ailment in felines, often resulting from poor food quality or sudden environmental changes, which can disrupt intestinal homeostasis. The primary symptom is diarrhea, and in some cases, it may be accompanied by severe inflammatory bowel disease.

Currently, the primary treatment for bacterial-induced intestinal disorders and inflammatory bowel disease (IBD) in felines involves the administration of antibiotics. The objective is to eradicate pathogenic bacteria, thereby facilitating the restoration of intestinal homeostasis. However, antibiotics exhibit a broad-spectrum bactericidal effect, which means they not only eliminate harmful bacteria but also eliminate beneficial microflora. Consequently, the duration of the treatment and the complete recovery period are often extended. Moreover, excessive or prolonged use of antibiotics can lead to the development of antibiotic resistance, resulting in a gradual decline in the efficacy of the drugs. However, there are currently no alternative therapeutic agents that demonstrate superior efficacy.

Therefore, the prior art still needs improvement.

### SUMMARY

In view of the aforementioned deficiencies in the prior art, the objective of the present disclosure is to provide a strain of Enterococcus faecalis and a composite bacterial agent, with the aim of improving therapeutic efficacy for intestinal disorders and inflammatory bowel disease in felines.

In order to achieve the above objective, the present disclosure adopts the following technical solutions:

A strain of Enterococcus faecalis, which has been deposited at Guangdong Microbial Culture Collection Center on Oct. 13, 2022, with the deposit number GDMCC No. 62869.

A composite bacterial agent includes Bifidobacterium longum, Enterococcus faecium and the above-mentioned Enterococcus faecalis.

An application of the Enterococcus faecalis as described above in a preparation of a drug for inhibiting an expression of inflammatory factors in intestinal cells.

An application of the Enterococcus faecalis as described above in a preparation of a drug for treating intestinal disorders in felines.

An application of the Enterococcus faecalis as described above in a preparation of a drug for treating inflammatory bowel disease in felines.

An application of the composite bacterial agent as described above in a preparation of a drug for treating intestinal disorders in felines.

An application of the composite bacterial agent as described above in a preparation of a drug for treating inflammatory bowel disease in felines.

### Beneficial Effects:

The present disclosure provides a strain of Enterococcus faecalis that can effectively treat bacterial-induced intestinal disorders or inflammatory bowel disease in felines, with a shorter treatment course compared to antibiotic drug treatments.

The present disclosure also provides a composite bacterial agent including Bifidobacterium longum, Enterococcus faecalis, and Enterococcus faecium. Through the synergistic effect of the three bacterial strains, the composite bacterial agent not only effectively treats intestinal disorders in felines but also further enhances the therapeutic efficacy for inflammatory bowel disease in felines.

### BRIEF DESCRIPTION OF THE DRAWINGS

FIG. 1 shows cytotoxicity test results of Enterococcus faecalis HHP003.

FIG. 2 shows expression levels of inflammatory factors in HT29 cells in cell experiments.

FIG. 3 shows a fecal score chart.

FIG. 4 shows an IgA content in a blood of felines in each group after 30 days of treatment with the drug or composite bacterial agent.

FIG. 5 shows an IgG content in a blood of felines in each group after 30 days of treatment with the drug or composite bacterial agent.

FIG. 6 shows an IgM content in a blood of felines in each group after 30 days of treatment with the drug or composite bacterial agent.

FIG. 7 shows a fecal scoring criteria.

### DETAILED DESCRIPTION OF EMBODIMENTS

The present disclosure provides a strain of Enterococcus faecalis, a composite bacterial agent and applications thereof. To make the objective, technical solution, and effect of the present disclosure clearer and more explicit, the following detailed description is provided with reference to the accompanying drawings and embodiments. It should be understood that the embodiments described herein are only intended to explain the present disclosure and are not intended to limit the present disclosure.

It should be noted that in the following embodiments, Enterococcus faecalis HHP003, which is the same as Enterococcus faecalis A-A-2 with the deposit number GDMCC No. 62869, was deposited on Oct. 13, 2022, at the Guangdong Microbial Culture Collection Center (GDMCC);

Bifidobacterium longum HHP004, which is the same as Bifidobacterium longum A-1-12 with the deposit number GDMCC No. 62866, was deposited on May 9, 2023, at the Guangdong Microbial Culture Collection Center (GDMCC);

Enterococcus faecium HHP005, which is the same as Enterococcus faecium M-3 with the deposit number GDMCC No. 62870, was deposited on Oct. 13, 2022, at the Guangdong Microbial Culture Collection Center (GDMCC).

### Embodiment 1

Human colon cancer cells HT29 were used, and the experiment was divided into five groups: the cell culture medium well (blank control well), the 37° C. treated control cell well (control well), the Enterococcus faecalis HHP003 treated well, the LPS treated cell well (treatment sample well), and the LPS+Enterococcus faecalis HHP003 treated well. The treatment methods for each group were detailed to in Embodiment 1.

After the treatment, LDH detection working solution was added, mixed well, and incubated at room temperature (approximately 25° C.) in the dark for 30 minutes, and the absorbance was then measured at 490 nm. The calculation formula is: cytotoxicity or LDH enzyme activity=(absorbance of the treated sample-absorbance of the sample control well)/(absorbance of the maximum enzyme activity of the cells−absorbance of the sample control well)×100.

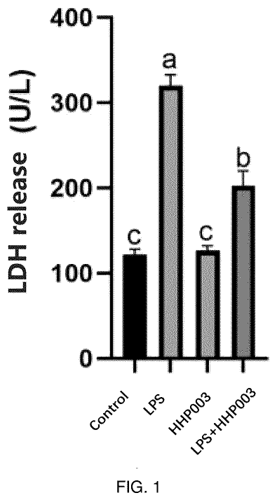

As shown in FIG. 1, the LDH production of HT29 cells is significantly increased after LPS treatment, indicating that LPS causes damage to HT29 cells; however, the LDH production of HT29 cells treated with Enterococcus faecalis HHP003 does not show a significant change, indicating that Enterococcus faecalis HHP003 is non-toxic to HT29 cells.

Additionally, the LDH content in the LPS+Enterococcus faecalis HHP003 group is significantly lower than that in the LPS group, indicating that the addition of Enterococcus faecalis HHP003 can significantly reduce the cell damage caused by LPS.

### Embodiment 2

### 2.1 Cell Experiments

### 2.1.1 Culture of Human Colon Cancer Cells HT29

Human colon cancer cells HT29 were cultured in RPMI1640 medium (HyClone, UT, USA) at 37° C. in a humidified 5% CO2 incubator, the medium was supplemented with 10% FBS (Gibco, Grand Island, NE, USA) and 1% antibiotics (100 mg/mL of streptomycin and 100 U/mL of penicillin). The HT29 cells were seeded at a density of 5×10⁵ cells per well in 12-well cell culture plates.

2.1.2 Co-Culture of Enterococcus faecalis HHP003 and HT29 Cells

The experiment was set up with four groups: the control group (Control), the HHP003 group, the LPS group, and the LPS+HHP003 group;

The procedures for the control group were as follows: HT29 cells were cultured without any additional treatment:

The procedures for the HHP003 group were as follows: when the HT29 cells reached 80% confluence, they were incubated in cell culture medium containing Enterococcus faecalis HHP003 (bacterial concentration of 1×10⁷ CFU/mL) for 6 hours;

The procedures for the LPS group were as follows: when the HT-29 cells reached 80% confluence, 10 μg/mL LPS was added, and the cells were incubated for 12 hours;

The procedures for the LPS+HHP003 group were as follows: when the HT-29 cells reached 80% confluence, they were first incubated in cell culture medium containing Enterococcus faecalis HHP003 (bacterial concentration of 1×10⁷ CFU/mL) for 6 hours. Subsequently, 10 μg/mL LPS was added, and the cells were treated for an additional 12 hours;

After the treatment in each group, the cells were washed three times with PBS, and then the HT29 cells were lysed using RNAiso 170Plus (Takara, Dalian, China) to extract RNA.

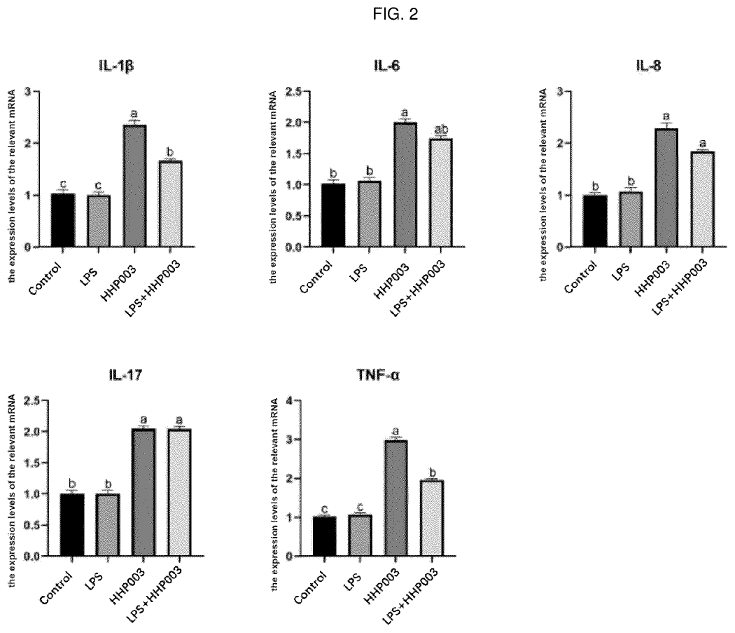

2.2 The expression levels of inflammatory cytokine genes were measured, and the corresponding results are shown in FIG. 2. The expression levels of inflammatory cytokines in the Control group and the HHP003 group are similar, with no significant differences, indicating that the use of Enterococcus faecalis HHP003 does not cause an upregulation of inflammatory cytokine expression in human colon cancer cells.

Compared to the Control group, the LPS group shows a significant upregulation in the gene expression of IL-1B, IL-6, IL-8, IL-17, TNF-α.

Compared to the LPS group, the LPS+HHP003 group shows a downregulation in the gene expression of IL-1B, IL-6, IL-8, and TNF-α, with significant differences in the expression levels of IL-1B and TNF-α. This indicates that co-culturing Enterococcus faecalis HHP003 with HT29 cells can effectively inhibit the upregulation of inflammatory factors induced by LPS and has the potential to achieve better therapeutic effects when used in combination with other drugs.

### Embodiment 3

### 3.1 Animal Experiments

Felines were randomly divided into four groups, with six felines in each group, the groups were as follows:
Control group (Group N): no additional treatment;
Drug group (Group A): From day 1 to day 8, the felines were continuously challenged by administering E. coli powder capsules once daily, with a dose of 1×10⁹ CFU per kilogram of body weight; from day 9 to day 30, the felines were given amoxicillin-clavulanate potassium, once daily, at a dose of 50 mg (1 tablet) each time;
Single strain probiotic group (Group 1S): From day 1 to day 8, the felines were continuously challenged in the same manner as the Drug group; from day 9 to day 30, the felines were given Enterococcus faecalis HHP003, with a dose of 1×10⁹ CFU per kilogram of body weight;
Three strains probiotic group (Group 3S): From day 1 to day 8, the felines were continuously challenged in the same manner as the Drug group; from day 9 to day 30, the felines were given a combination of Enterococcus faecalis HHP003, Bifidobacterium longum HHP004, and Enterococcus faecium HHP005, with a dose of 1×10⁹ CFU per kilogram of body weight for each strain;
the felines were bled on day 0, day 8, and day 30 to measure the relevant blood indicators;
the feces of the felines were scored;
a fecal scoring criteria are based on the following reference: Evaluation of canned therapeutic diets for the management of the felines with naturally occurring chronic diarrhea, Journal of Feline drug and Surgery, 14 (10) 669-677, DOI: 10.1177/1098612X12446906.

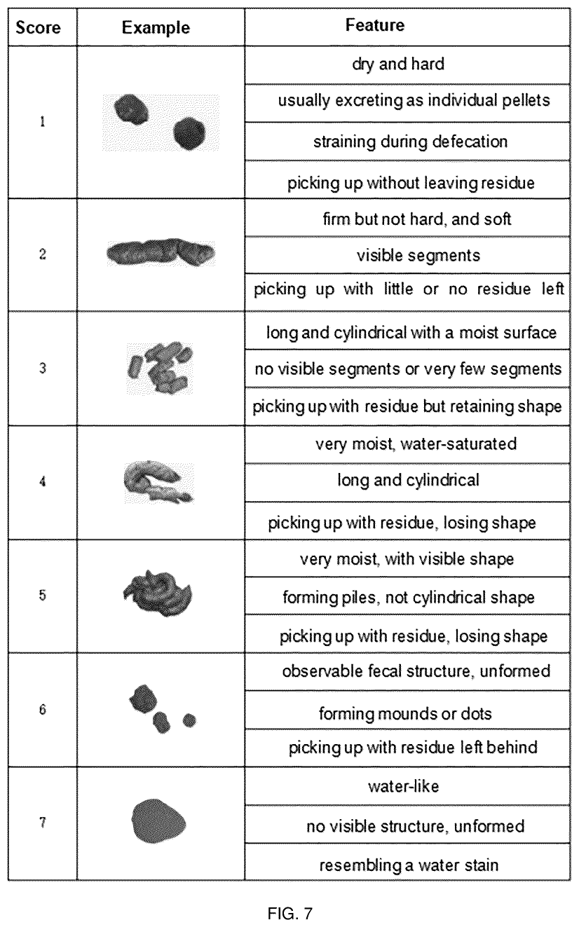

The fecal scoring criteria were shown in FIG. 7. In the scoring criteria of FIG. 7, the closer the score is to 2 points, the better the health status of the cat is.

3.2 The results of the fecal tests are as follows:

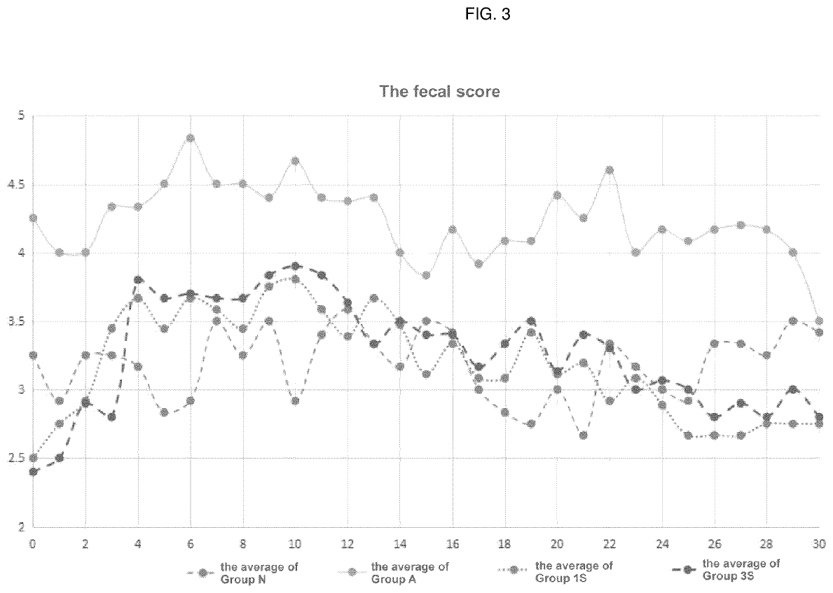

Refer to FIG. 3, which shows the daily trend of average fecal scores for each group of the felines. For the Control group (Group N), which does not undergo any challenge treatment and is not given probiotics or antibiotics, the fecal scores of Group N in FIG. 1 remain stable between 2.5 and 3.5;
the fecal scores of the Drug group (Group A) gradually increase after the challenge treatment. After discontinuing the challenge and administering antibiotics, there is a significant downward trend in fecal scores from day 10 to day 15. However, there is a relapse on days 20 and 22, indicating that while the use of antibiotics can help to repair intestinal homeostasis in the short term, long-term use may lead to the development of bacterial resistance;
the fecal scores of the Single strain probiotic group (Group 1S) also gradually increase after the challenge treatment, and the increase is significantly greater than that of the Drug group (Group A), indicating that the felines in this group are more sensitive to the challenge. After discontinuing the challenge and administering Enterococcus faecalis HHP003, the fecal scores of Group 1S continuously decrease from day 10 to day 25 and then stabilize between 2.5 and 3. Additionally, during the same period, the rate of decrease in fecal scores for Group 1S is significantly faster than that of Group A, suggesting that Enterococcus faecalis HHP003 is more effective in treating intestinal disorders in the felines compared to the Drug group (Group A);
the fecal scores of the Three strains probiotic group (Group 3S) also gradually increase after the challenge treatment. After discontinuing the challenge and administering Enterococcus faecalis HHP003, Bifidobacterium longum HHP004, and Enterococcus faecium HHP005, the trend in fecal scores is similar to that of the Single probiotic group (Group 1S). This indicates that the combined use of Enterococcus faecalis HHP003, Bifidobacterium longum HHP004, and Enterococcus faecium HHP005 is more effective in treating intestinal disorders in the felines compared to the Drug group (Group A).

3.3 IgA Content in the blood of the felines on day 30 after feeding with drugs or probiotics

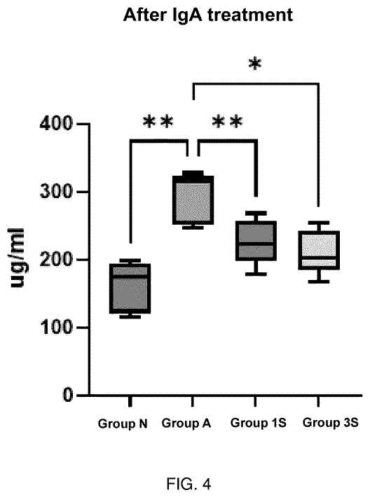

Refer to FIG. 4, which shows the IgA content in the blood of each group of the felines on day 30. From FIG. 4, it can be seen that the Control group (Group N), which does not undergo any challenge treatment or receive drugs or probiotics, has IgA levels within the normal range;
the IgA content in the Drug group (Group A) is very high, and there is a significant difference compared to the Control group (Group N), indicating that although the Drug group (Group A) improved the intestinal disorders in the felines, the inflammation caused by the challenge is not effectively eliminated;
the IgA content in the Single strain probiotic group (Group 1S) is lower than that in the Drug group (Group A), and there is no significant difference compared to the Control group (Group N), indicating that Enterococcus faecalis HHP003 can effectively eliminate the inflammation caused by the challenge and treat inflammatory bowel disease;
the IgA content in the Three strains probiotic group (Group 3S) is lower than that in the Drug group (Group A), and there is no significant difference compared to the Control group (Group N), indicating that the combination of Enterococcus faecalis HHP003, Bifidobacterium longum HHP004, and Enterococcus faecium HHP005 can also effectively eliminate the inflammation caused by the challenge and treat inflammatory bowel disease.

3.4 IgG content in the blood of the felines on day 30 after feeding with drugs or probiotics

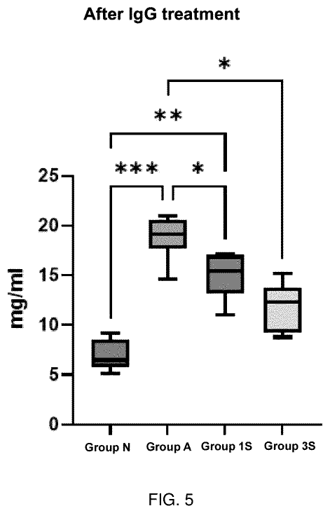

Refer to FIG. 5, which shows the IgG content in the blood of each group of the felines on day 30. From FIG. 5, it can be seen that the Control group (Group N), which do not undergo any challenge treatment or receive drugs or probiotics, has IgG levels within the normal range;
the IgG content in the Drug group (Group A) is very high, and there is an extremely significant difference compared to the Control group (Group N), indicating that the inflammatory issues in the felines of the Drug group (Group A) are still very evident, and the use of antibiotics can not effectively eliminate the inflammation caused by the challenge;
the IgG content in the Single strain probiotic group (Group 1S) is lower than that in the Drug group (Group A), and there is a significant difference compared to the Drug group (Group A), indicating that Enterococcus faecalis HHP003 can to some extent, improve the inflammation caused by the challenge and treat inflammatory bowel disease;
the IgG content in the Three strains probiotic group (Group 3S) is significantly different from that in the Drug group (Group A), and it is lower than that in the Single strain probiotic group (Group 1S), indicating that the combination of Enterococcus faecalis HHP003, Bifidobacterium longum HHP004, and Enterococcus faecium HHP005 has better therapeutic effects compared to the single probiotic group.

3.5 IgM content in the blood of the felines on day 30 after feeding with drugs or probiotics

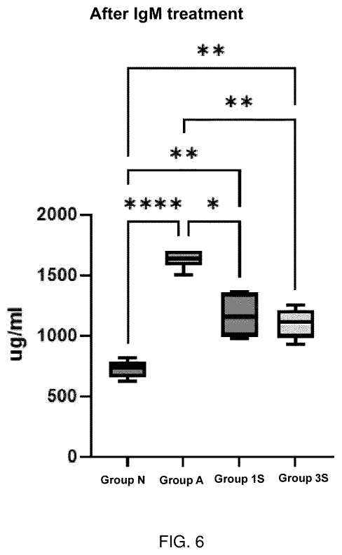

Refer to FIG. 6, which shows the IgM content in the blood of each group of the felines on day 30. From FIG. 6, it can be seen that the control group (Group N), which does not undergo any challenge treatment or receive drugs or probiotics, has IgM levels within the normal range;
the IgM content in the Drug group (Group A) is very high, and there is an extremely significant difference compared to the Control group (Group N), indicating that the inflammatory issues in the felines of the Drug group (Group A) are still very evident, and the use of antibiotics is not effective in treating the inflammation caused by the challenge;
the IgM content in the Single strain probiotic group (Group 1S) is lower than that in the Drug group (Group A), and there is a significant difference compared to the Drug group (Group A), indicating that Enterococcus faecalis HHP003 can improve the inflammation caused by the challenge and treat inflammatory bowel disease;
the IgM content in the Three strains probiotic group (Group 3S) is lower than that in the Drug group (Group A), and there is a significant difference compared to the Drug group (Group A), indicating that the combination of Enterococcus faecalis HHP003, Bifidobacterium longum HHP004, and Enterococcus faecium HHP005 can improve the inflammation caused by the challenge and treat inflammatory bowel disease.

In summary, antibiotic treatment for feline intestinal disorders has some effect, but the treatment duration is long, and the effects are not obvious. Additionally, antibiotics are not effective in treating inflammatory bowel disease in the felines. The probiotic formulations of Group 1S and Group 3S in the present disclosure not only treat feline intestinal disorders but also show, through fecal scores and immunoglobulin indicators, that they have significantly better therapeutic effects compared to the antibiotic treatment (Group A). The probiotic formulations have good application prospects and practical value.

Furthermore, a comprehensive comparison of the fecal scores and the levels of IgA, IgG, and IgM between Group 1S and Group 3S reveals that the overall therapeutic effect of Group 3S is better than that of Group 1S, although the magnitude and trend of the decrease in fecal scores are similar between Group 3S and Group 1S; however, a comprehensive comparison of the levels of IgA, IgG, and IgM show that on day 30, the levels of the three main immunoglobulins in the 3S group were all lower than those in the 1S group, indicating that the 3S group has a better effect in treating inflammatory bowel disease. This suggests that further combining the use of Bifidobacterium longum and Enterococcus faecium with Enterococcus faecalis can improve the efficacy of treatment.

It is understood that to those skilled in the art, equivalent substitutions or modifications can be made based on the technical solutions and inventive concepts of the disclosure, and all such modifications or substitutions should fall within the scope of protection of the appended claims of the present disclosure.

What is claimed is:

1. A strain of Enterococcus faecalis, wherein the Enterococcus faecalis has been deposited at Guangdong Provincial Microbiological Culture Collection Center on Oct. 13, 2022, with deposit number GDMCC No. 62869.

2. A composite bacterial agent, comprising Bifidobacterium longum, Enterococcus faecium and the Enterococcus faecalis according to claim 1.

3. An application of the Enterococcus faecalis according to claim 1, in a preparation of a drug for inhibiting an expression of inflammatory factors in intestinal cells, a drug for treating intestinal disorders in felines, and a drug for treating inflammatory bowel disease in felines.

4. An application of the composite bacterial agent according to claim 2, in a preparation of a drug for treating intestinal disorders in felines and a drug for treating inflammatory bowel disease in felines.
